(19) United States
(12) Patent Application Publication
(10) Pub. No.: US 2010/0143897 A1
(43) Pub. Date: Jun. 10, 2010

(54) Materials and Methods for Assaying for Glyoxylate

(75) Inventors: David J. Merkler, Valrico, FL (US); Sarah E. Carpenter, Temple Terrace, FL (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: University of South Florida, Tampa, FL (US)

(21) Appl. No.: 12/066,922

(22) PCT Filed: Sep. 15, 2006
(86) PCT No.: PCT/US06/36147

§ 371 Date: Dec. 22, 2009

Related U.S. Application Data
(60) Provisional application No. 60/717,657, filed on Sep. 16, 2005.

(51) Int. Cl. C12Q 1/68; G01N 33/00; C12Q 1/32; C12Q 1/26; C12Q 1/28

(52) U.S. Cl. 435/6; 436/128; 435/26; 435/25; 435/28

(57) ABSTRACT

The subject invention concerns enzyme-based methods for detecting and assaying for glyoxylate. In particular, the invention is directed to methods for assaying for glyoxylate produced by the reaction of peptidylglycine α-amidating monooxygenase (PAM). The subject invention also concerns methods for assaying for the enzyme peptidylglycine α-amidating monooxygenase. The detection of glyoxylate using the present invention is indicative of the presence of PAM. The subject invention also concerns methods for screening for peptide hormones and any N-acyl-glycine or N-aryl-glycine conjugated molecule.

This application claims the benefit of U.S. Provisional Application Ser. No. 60/717,657, filed Sep. 16, 2005, which is hereby incorporated by reference herein in its entirety, including any figures, tables, nucleic acid sequences, amino acid sequences, and drawings.

This invention was made with government support under the National Institute of Health SBIR grant number 1-R43-DK063812-01. The government has certain rights in the invention.

### BACKGROUND OF THE INVENTION

C-Terminal amidation of glycine extended prohormones is a post-translational modification necessary for the activity of amidated peptide hormones. Amidated peptide hormones are an important class of hormones found in mammals, insects, and cnidarians. The discovery of amidated hormones has been severely hindered by the lack of an assay specific to this class of bioactive hormones. The formation of all amidated peptide hormones is dependant upon the activity of Peptidylglycine α-Amidating Monooxygenase (PAM). For each glycine extended precursor activated by PAM an α-amidated peptide and glyoxylate result. The general reaction is shown below in Scheme 1:

Peptide-(glycine)→peptide-NH₂+glyoxylate Scheme 1

Glyoxylate (HCO—COO⁻), a product of the reaction shown in Scheme 1, is a metabolite synthesized and catabolized by both vertebrates and invertebrates (Gragera et al., 2000). Calcium oxalate is the major constituent of kidney stones (Asplin, 2002) and approximately 50-60% of urinary oxalate (⁻OOC—COO⁻) is derived from the enzymatic oxidation of glyoxylate (HCO—COO⁻) (Williams et al., 1989). As a consequence of the metabolic importance and role of glyoxylate in kidney stone formation, a number of assays have been developed for glyoxylate. Existing assays for the determination of glyoxylate include colorimetric methods (Albrecht et al., 1962; Soda et al., 1973; Bongers et al., 1992; Kramer et al., 1959; Vogels et al., 1970), fluorometric methods (Spikner et al., 1962; Zarembski et al., 1965), the iodometric or potentiometric titration of the bisulfite adduct (McFadden et al., 1960), and the use of capillary electrophoresis with direct UV detection (Nishijima et al., 2001; Garcia et al., 2001). Generally, these are insensitive, nonspecific, or both. Such drawbacks have been overcome by the separation and quantification of the colored or fluorescent glyoxylate derivative by HPLC (Bongers et al., 1992; Funai et al., 1986; Mentasi et al., 1987; Petrarulo et al., 1988; Lange et al, 1994). Thus, there remains a need in the art for a rapid, specific, sensitive assay for glyoxylate.

### BRIEF SUMMARY OF THE INVENTION

The subject invention concerns enzyme-based methods for detecting and assaying for glyoxylate. Assays utilizing several different enzymes for assaying for glyoxylate are provided herein. Exemplified herein are assays wherein detection is accomplished using spectrophotometry, fluorescence, or luminescence.

The subject invention also concerns methods for assaying for the enzyme peptidylglycine α-amidating monooxygenase (PAM). The detection of glyoxylate using the present invention is indicative of the presence of PAM. PAM is known to oxidatively cleave glycine-extended peptide and fatty acid substrate prohormones to the amidated product and glyoxylate in an equal ratio. Glycine-extended prohormones are relatively inactive prior to

PAM dependent amidation. Moreover, PAM regulates hormonal activity by amidating glycine extended substrates, therefore assaying for PAM activity by quantifying glyoxylate allows one to not only test PAM activity but to also assay a wide variety of glycine extended substrate derivatives.

The subject invention also concerns methods for screening for peptide hormones and any N-acyl-glycine or N-aryl-glycine conjugated molecule. The detection of glyoxylate using the present invention is indicative of the presence of PAM. The presence of PAM is likewise indicative that an α-amidated peptide is also being produced. The subject invention provides a means for the discovery of novel hormones that regulate proper mammalian function.

### BRIEF DESCRIPTION OF THE DRAWINGS

FIG. 1 shows spectrophotometric malate synthase/malate dehydrogenase (MS/MD) enzyme-linked colorimetric assay for glyoxylate. Glyoxylate is measured by the malate synthase/malate dehydrogenase-dependent formation of an intensely colored formazan (1=malate synthase, 2=malate dehydrogenase, and PMS=phenazine methosulfate). PMS serves to shuttle electrons from NADH to the tetrazolium (MTS).

FIGS. 2A and 2B show glyoxylate-dependent oxidation of MTS. The increase in absorbance obtained using glyoxylate (FIG. 2A) and that obtained by the base-catalyzed dealkylation of α-hydroxyhippurate to benzamide and glyoxylate (FIG. 2B). The data points are the average of 3-10 determinations and the error bars represent the standard deviation of the measurements.

FIG. 3 shows lactate dehydrogenase-glycolate oxidase, glycolate oxidase reaction (LD-GO, GO). Glyoxylate is measured by the enzyme dependant oxidative coupling of two compounds which produce an indamine dye measurable at 590 nm. The PAM substrate dns-YV-Gly was collected by Preparative HPLC, reacted with PAM, and the product DNS-YV—NH₂ was quantified by HPLC. Concentration of glyoxylate was determined from the 1:1 molar ratio of dns-YV—NH₂:glyoxylate. Standard Curve analysis of PAM produced glyoxylate is shown in FIG. 4 to match the literature cited Indamine extinction coefficient. Slope=0.05000

FIG. 4 shows analysis of glyoxylate/glycine-extended peptide by the LD-GO, GO assay. This graph demonstrates that the dansylated tripeptide Tyr-Val-Gly can be quantitatively measured by production of hydrogen peroxide according to the literature value of 0.05300M⁻cm⁻.

FIG. 5 shows the glyoxylate reductase spectrophotometric assay for glyoxylate. The enzyme activity of glyoxylate reductase consumes a stoichiometric quantity of NADPH to glyoxylate, the loss of NADPH can be measured at 340 nm. The PAM substrate dns-TYGly was collected by Preparative HPLC, reacted with PAM, and the product DNS-TVNH₂ was quantified by HPLC. Concentration of glyoxylate was determined from the 1:1 molar ratio of dns-TYNH₂: glyoxylate. Standard Curve analysis of PAM produced glyoxylate is shown in FIG. 6 to match the literature cited NADPH extinction coefficient. Slope=0.0063.

FIG. 6 shows stoichiometric detection of glyoxylate and an α-amidated peptide. This graph demonstrates the ability of this glyoxylate assay to be applicable to the PAM assay system for the identification of α-amidated hormones. The literature value of NADPH consumption is 0.0062M⁻cm⁻.

FIGS. 7A and 7B show fluorescent enzymatic assays for glyoxylate. Both enzymatic assays utilize the oxidation of Amplex Red as the fluorescent analyte for detection of glyoxylate. The oxidation of Amplex Red is dependant upon the production of hydrogen peroxide. The reaction in FIG. 7A uses glycolate oxidase; The reaction in FIG. 7B uses glyoxal oxidase. The PAM substrate dns-YV-Gly was collected by Preparative HPLC, reacted with PAM, and the product dns-YV—NH₂ was quantified by HPLC. Concentration of glyoxylate was determined from the 1:1 molar ratio of dns-YV—NH₂: glyoxylate. Standard Curve analysis of PAM produced glyoxylate is shown in FIGS. 8A and 8B for each enzyme assay, and matches the standard curve for H₂O₂.

FIGS. 8A and 8B show stoichiometric detection of glyoxylate by fluorescence. Both assays show linear detection of both pure glyoxylate and PAM produced glyoxylate. The PAM substrate dansyl-Tyr-Val-Gly was independently quantified and used to quantitative PAM produced glyoxylate which was detected in this assay. Results from the reaction of FIG. 7A are shown in FIG. 8A and results from the reaction of FIG. 7B are shown in FIG. 8B.

FIG. 9 shows description of luminescent assay for glyoxylate. Both Glyoxal Oxidase and Glycolate Oxidase can be used for the luminescent detection assay. For detection of PAM produced glyoxylate the first two sets are necessary, and can be eliminated for detection of glyoxylate alone. The PAM substrate dns-YV-Gly was collected by Preparative HPLC, reacted with PAM, and the product dns-YV—NH₂ was quantified by HPLC. Concentration of glyoxylate was determined from the 1:1 molar ratio of dns-YV—NH₂: glyoxylate. Standard Curve analysis of PAM produced glyoxylate is shown in FIG. 11, and matches the standard curve for H₂O₂/glyoxylate.

FIG. 10 shows reaction mechanism for the production of light from luminol. Oxidation to the excited state of luminol is proportional to the quantity of hydrogen peroxide.

FIG. 11 shows glyoxylate oxidase luminescent detection of glyoxylate. This data demonstrates the ability of the luminescent assay to detect the presence of a glycine-extended peptide via glyoxylate.

FIG. 12 shows flow chart of independent analysis of accumulated peptides by both luminescence, and MALDI-TOF.

FIGS. 13A and 13B show HPLC fractions collected and assayed for glyoxylate. FIG. 13A shows detection of spiked cell culture spiked mJP-Gly (2.5 nmoles) by luminescent analysis of fractions for glyoxylate. FIG. 13B shows detection of mJP-Gly accumulated in cell culture by the presence of a PAM inhibitor, by the luminescent assay for PAM. Identification of mJP-Gly via glyoxylate in the same fraction is conclusive that the glyoxylate observed is derived from the PAM dependant conversion of mJP-Gly to α-amidated-mJP and glyoxylate.

FIGS. 14A-14D show identification of glyoxylate positive fraction for the presence of mJP-Gly. Fractions 31 from both a spiked sample (FIGS. 14C and 14D) and non-spiked sample (FIGS. 14A and 14B) were assayed for the presence of mJP by MALDI-TOF Mass Spectrometry. Identification of the glycine-extended form demonstrates that indeed PAM was inhibited in cell culture, and the glyoxylate is coincident with the peptides analyzed by Mass Spectrometry.

FIG. 15 shows PAM reaction scheme and reaction for removal of ascorbate prior to glyoxylate assay.

### DETAILED DISCLOSURE OF THE INVENTION

The subject invention concerns enzyme-based methods for detecting and assaying for glyoxylate. Glyoxylate is a molecule of interest to the scientific community as its in vivo production is signature of many health issues. Likewise, glyoxylate is involved in several plant biochemical pathways namely the “glyoxylate cycle”, and therefore analyzing glyoxylate concentration with this new technology will be of importance in the fields of plant biochemistry, botany, and horticulture. In one embodiment, methods of the invention can be used for assaying for glyoxylate produced by the reaction of peptidylglycine α-amidating monooxygenase (PAM).

The subject invention also concerns methods for assaying for the enzyme peptidylglycine α-amidating monooxygenase. The detection of glyoxylate using the present invention is indicative of the presence of PAM. PAM is the key enzyme in the regulation of over 50% of all known hormones, and has been widely studied by the scientific community based on its extremely important physiological role. PAM is known to oxidatively cleave glycine-extended peptides and fatty acid substrate prohormones to the amidated product and glyoxylate in an equal ratio. Glycine-extended prohormones are relatively inactive prior to PAM dependent amidation. Moreover, PAM regulates hormonal activity by amidating glycine extended substrates. Therefore, assaying for PAM activity by quantifying glyoxylate allows one to not only test for PAM activity but to also assay for a wide variety of glycine extended substrate derivatives.

The subject invention also concerns methods for screening for glycine extended molecules, such as peptides and hormones and any N-acyl-glycine or N-aryl-glycine conjugated molecule. In one embodiment, a sample to be tested is contacted with PAM, wherein if a glycine extended molecule is present, then the PAM acts on the molecule resulting in the production of glyoxylate. The glyoxylate can then be assayed for using any of the methods of the present invention. The detection of glyoxylate using the present invention is indicative of the presence of PAM. The presence of PAM is likewise indicative that an α-amidated peptide is also being produced. Defining tissues that have high levels of PAM activity provides researchers with a place to search for novel peptide hormone substrates. Currently, there exists no efficient and sensitive technique for the discovery of novel peptide hormones. The subject invention provides a means for the discovery of novel hormones that regulate proper mammalian function. Thus, the present invention provides a novel series of assays specific for the discovery of numerous unidentified amidated hormones. The assays exploit this very unique biosynthetic pathway for the formation of the amidated peptides.

As exemplified herein, a series of enzyme dependent assays for glyoxylate with detection by, for example, spectrophotometry, fluorescence, and luminescence, have been developed. The assays of the present invention can use any suitable detection means and are not limited to those means specifically exemplified herein. Several areas can benefit from this technology ranging from the medical fields to research science. Three different spectrophotometric assays are exemplified herein, each of which utilizes different enzyme detection systems.

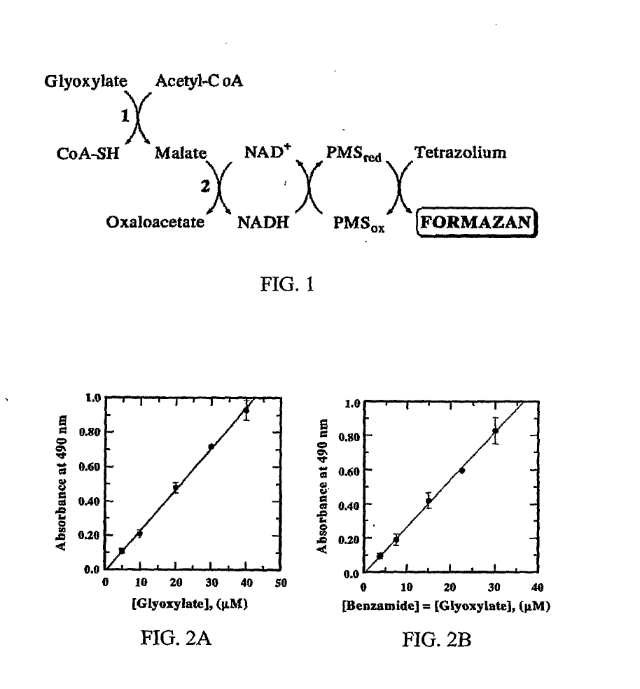

Methods for detecting glyoxylate in a sample comprise contacting the same with one or more reagents, such as an enzyme, that result in the production of a detectable reaction product if glyoxylate is present. The detectable reaction product can then be detected by any suitable means including, but not limited to, visual means, spectrophotometric means, fluorescent means, luminescent means, and the like. One embodiment of the present invention concerns an assay that utilizes malate synthase/malate dehydrogenase in which enzymatically oxidized glyoxylate and acetyl-CoA produce oxaloacetate with the concomitant reduction of NAD⁺ to NADH. A sample to be assayed for glyoxylate is contacted with Acetyl-CoA and malate synthase and malate dehydrogenase. The presence of glyoxylate in the sample results in oxaloacetate and NADH production. In one embodiment, NADH produced is then detected using phenazine methosulfate (PMS) and a tetrazolium compound, such as 3-(4,5-dimethylthiazol-2-yl)-5-(3-carboxymethoxylphenyl)-2-(4-sulfophenyl)-2H-tetrazolium, inner salt (MTS). NADH drives reduction of PMS which in turn drives the reduction of a tetrazolium compound (MTS) to produce an intensely colored reduced formazan with a VIS detection limit of approximately 5 nanomoles glyoxylate. A general reaction scheme for the assay is shown in FIG. 1 and results are shown in FIGS. 2A-2B.

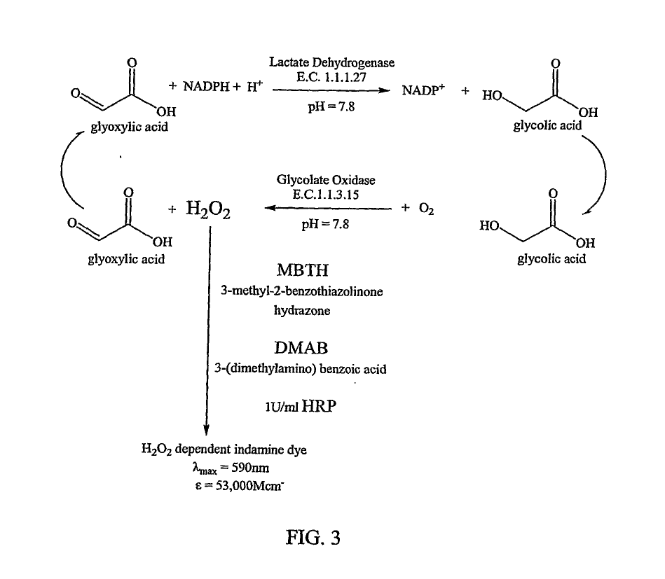

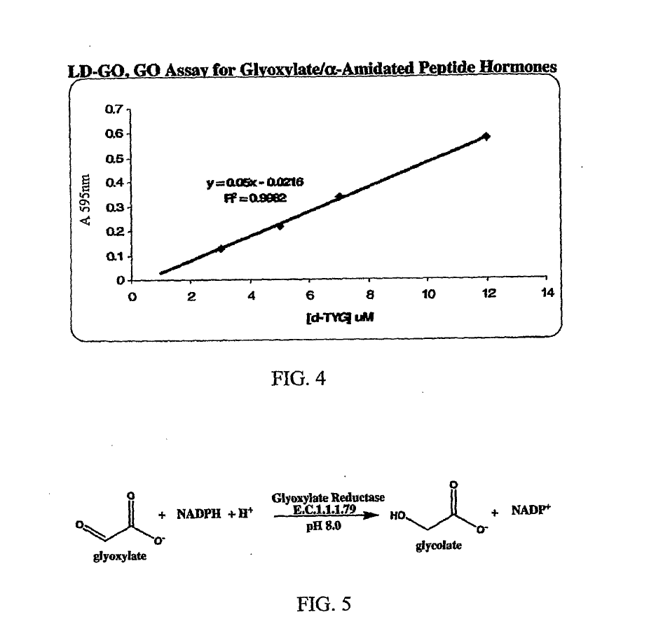

A further embodiment of the invention concerns an assay that utilizes lactate dehydrogenase/glycolate oxidase-glycolate oxidase (LD-GO,GO) which oxidizes glyoxylate to glycolate with a stoichiometric production of hydrogen peroxide. A sample to be assayed for glyoxylate is contacted with lactate dehydrogenase and glycolate oxidase. The hydrogen peroxide produced from the enzymatic reaction can then be detected by any of a variety of techniques. In one embodiment with spectrophotometric detection, an MBTH/DMAB-indamine dye detection system can be used based on its low detection limit for a spectrophotometric assay of 300 nmoles. A general reaction scheme for the lactate dehydrogenase/glycolate oxidase-glycolate oxidase assay is shown in FIG. 3 and results are shown in FIG. 4.

In another embodiment, the present invention concerns an assay that utilizes glycolate oxidase or glyoxal oxidase to produce oxalate and a stoichiometric amount of H₂O₂ from glyoxylate. The H₂O₂ produced from the reaction can then be detected by any of a variety of techniques. In one embodiment, a fluorescent-based detection method that utilizes Amplex Red and horseradish peroxidase are used to detect H₂O₂, wherein the Amplex Red is oxidized to the fluorescent molecule Resorufin. Other methods for detection of H₂O P₂ are known in the art and are contemplated within the scope of the present invention.

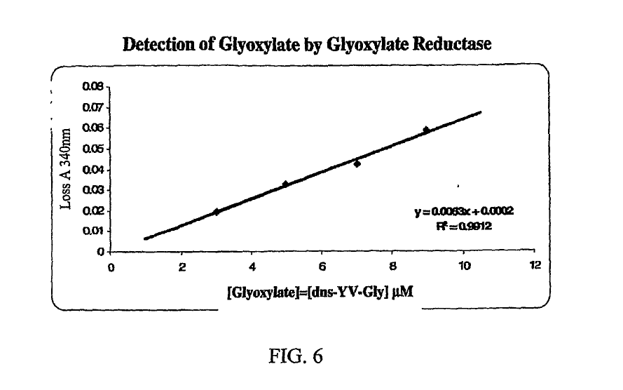

In a still further embodiment, the present invention concerns an assay that utilizes glyoxylate reductase in which glyoxylate is reduced to glycolate with the concomitant oxidation of NADPH to NADP⁺. Production of NADP⁺ results in a change in absorbance at 340 nm with a detection limit of 900 nmoles. A general reaction scheme for the assay is shown in FIG. 5 and results are shown in FIG. 6.

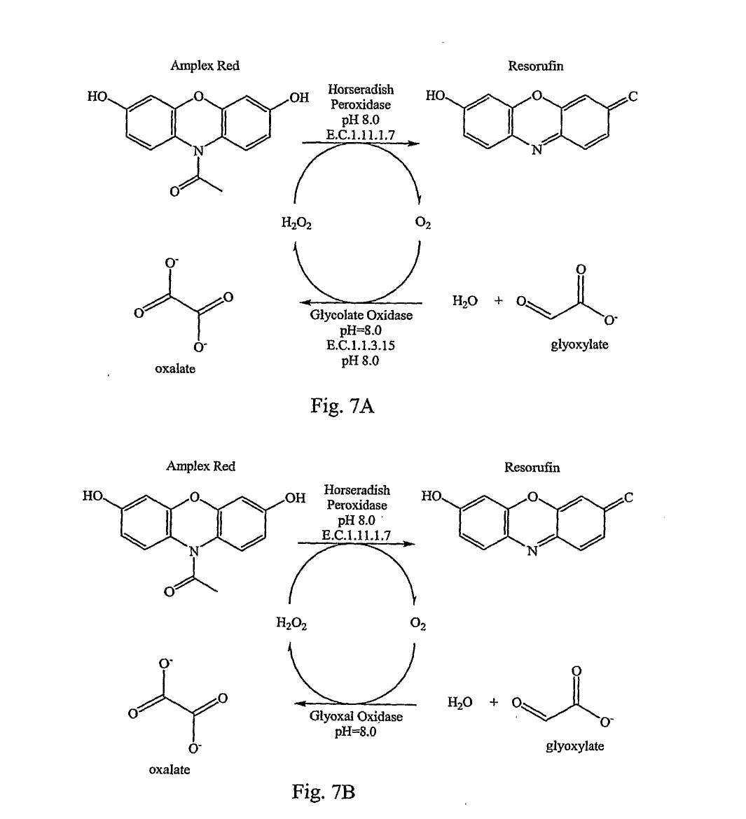

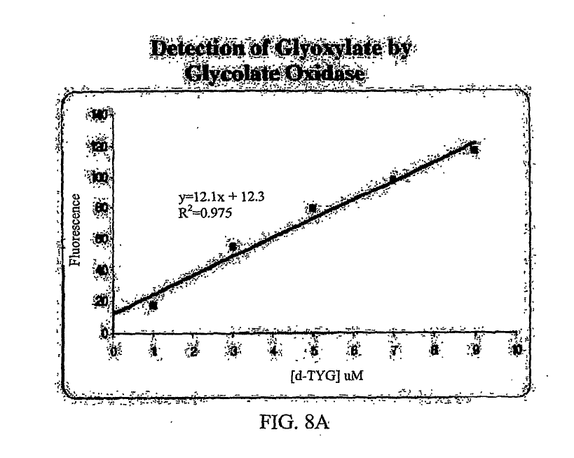

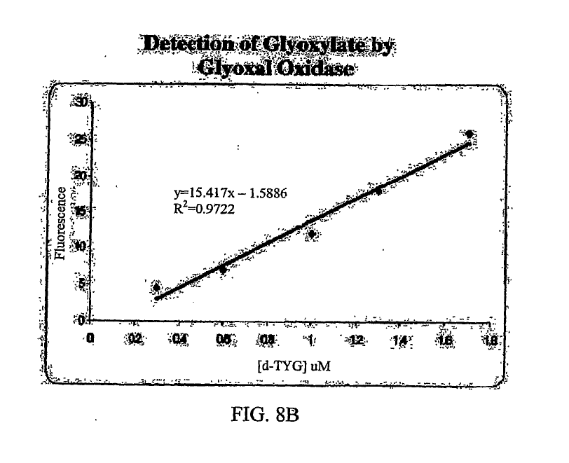

Fluorescence-based detection methods that can be used in the present invention are based on enzyme-dependant stoichiometric production of hydrogen peroxide to glyoxylate consumption. Hydrogen peroxide is detectable by a variety of techniques. In one embodiment, hydrogen peroxide is detected by reaction of a substrate, typically in a non-fluorescent state, in the presence of hydrogen peroxide to produce a fluorescent molecule. One assay exemplified herein utilizes Amplex Red, a non-fluorescent substrate for horseradish peroxidase which in the presence of hydrogen peroxide oxidizes to the highly fluorescent molecule Resorufin (see FIGS. 7A and 7B). Oxidation of Amplex Red is dependent upon the presence of hydrogen peroxide and this assay proved stoichiometric for the quantification of glyoxylate based on the chemistry of the chosen enzymatic reactions. To modify the assay for high through-put analysis, as well as sensitivity, the assay was modified to a microplate format with detection levels in the range of 10-30 pmole. Two enzymes were chosen for the fluorescent assay: glyoxal oxidase (the general reaction scheme is shown in FIG. 7B and results are shown in FIG. 8B) and glycolate oxidase (the general reaction scheme is shown in FIG. 7A and results are shown in FIG. 8A). Both enzymes produce stoichiometric quantities of hydrogen peroxide from glyoxylate. It is imperative that all FMN (flavin mononucleotide) be removed from the glycolate oxidase enzyme prior to glyoxylate analysis with Amplex Red. This molecule is oxidatively labile and will auto-oxidize in the presence of FMN. As all FMN must be removed from the glycolate oxidase enzyme, FAD (flavin adenine dinucleotide) is utilized as the flavin of choice for this reaction, as FAD also supports GO catalysis yet does not oxidize Amplex Red.

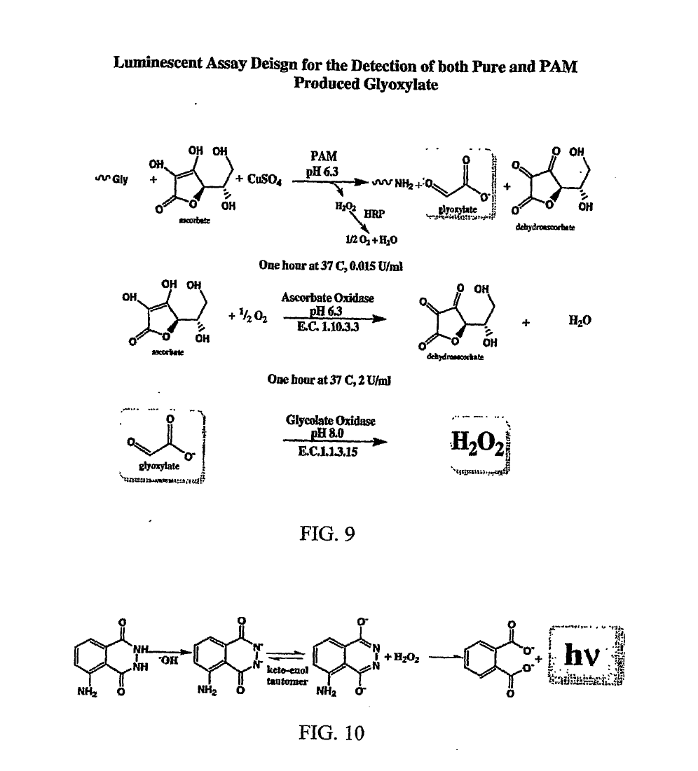

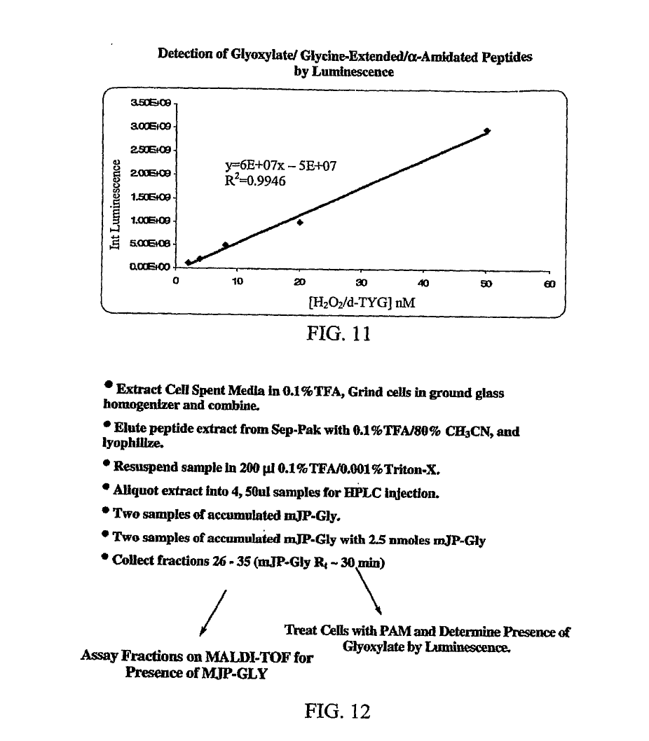

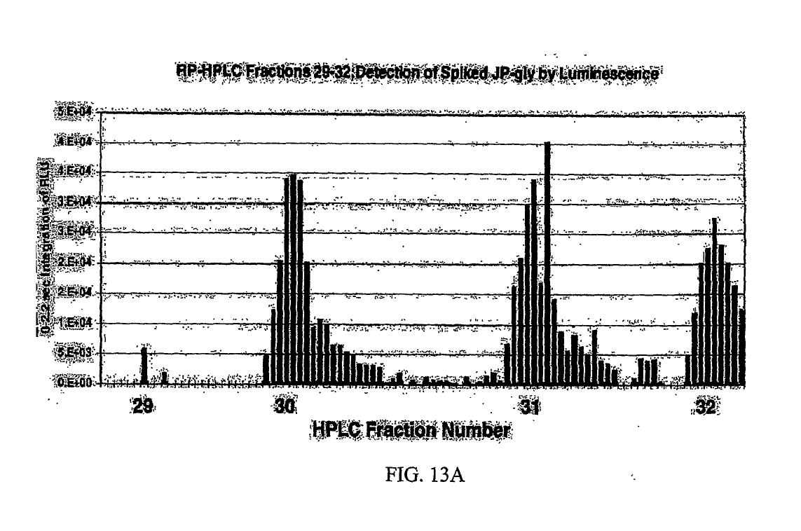

Luminescence based detection methods can also be used with the assays of the present invention and proved to be the most sensitive. An exemplified assay is based on the chemiluminescence of luminol. In the presence of an iron catalyst and a basic environment, luminol becomes excited into the triplet spin state in the presence of hydrogen peroxide. The relaxation of luminol back to the singlet state then releases a photon of light (see FIG. 10). The emission of light is concentration dependant, thereby affording a highly sensitive technique for analyzing hydrogen peroxide concentration in the femtomole region. Utilizing glycolate oxidase and/or glyoxal oxidase, a stoichiometric amount of hydrogen peroxide is produced (see FIG. 9), thus providing the most sensitive of all techniques exemplified herein for the quantification of glyoxylate. Results using a luminescent assay of the invention are shown in FIG. 11.

Assays of the present invention can be used to screen for the presence of an amidated peptide in a sample. In one embodiment, cells are grown, optionally in the presence of a PAM inhibitor, to accumulate glycine extended peptides. Cell extracts and/or spent media are prepared from the grown cells. Chromatographic techniques, such as HPLC, can then be used to fractionate the cell extracts and/or spent media samples. The HPLC fractions can then be treated with PAM. The accumulated glycine extended peptides are acted on by PAM to produce the amidated peptide plus glyoxylate. The PAM treated fractions (ascorbate can be removed) can then be assayed for the presence of the glyoxylate (produced by the PAM reaction) using any assay of the present invention. Fractions which contain glyoxylate are positive for a glycine extended peptide. Glycine extended peptides can be characterized by mass spectrometry to determine the identity of the amidated peptide.

Application of the assays of the invention to the quantification of PAM produced glyoxylate required two alterations of the PAM assay, as well as one change to the common glycolate oxidase assay which has previously been mentioned. Without these specific alterations the assays would be rendered non-stoichiometric and produce anomalous data. First, to address the changes to the PAM assay, a new method for enzyme protection due to hydroxyl radical formation produced during catalysis was necessary as the use of catalase has previously been the method of choice. Catalase catalyzes the disproportionate reaction of H₂O₂ to H₂O and O₂, and removal of hydrogen peroxide is detrimental to the assay methods. Horseradish peroxidase was found to both protect the PAM enzyme, and not interfere with hydrogen peroxide detection. Second, ascorbate is a reductant necessary for the PAM catalysis and was found to both severely inhibit the spectrophotometric, fluorescent, and luminescent assays, and the enzyme activity of glyoxal oxidase. An alternative reductant, catechol, proved more desirable as it is not an inhibitor for any of the assays described herein.

The subject invention also concerns kits comprising reagents for use in practicing the methods of the invention. In one embodiment, a kit comprises one or more of acetyl-CoA, malate synthase, malate dehydrogenase, phenazine methosulfate, and a tetrazolium compound such as MTS. In another embodiment, a kit comprises one or more of lactate dehydrogenase, glycolate oxidase, MBTH, DMAB, and a peroxidase, such as horseradish peroxidase (HRP). In a further embodiment, a kit comprises one or more of glycolate oxidase, glyoxal oxidase, a peroxidase such as HRP, a substrate that reacts in the presence of the peroxidase to produce a fluorescent molecule, such as Amplex Red, FAD, and FMN. In another embodiment, a kit comprises one or more of glyoxylate reductase. Any kit of the invention can also optionally comprise one or more of ascorbate, ascorbate oxidase, and catechol.

### Materials and Methods

Glyoxylate Assays

Spectrophotometric enzyme-linked assays for glyoxylate were initiated by the addition of malate synthase and malate dehydrogenase. The assay contained 100 mM TEA-HCl pH 7.8, 150 μM/8.25 μM MTS/PMS, 10 mM MgCl₂, 400 μM acetyl-CoA, 500 μM NAD⁺, 0-50 μM glyoxylate, 6 units/ml malate synthase, and 6 units/ml malate dehydrogenase in a final volume of 1 ml. The absorbance at 490 nm was measured after one hour incubation at 37° C. in the dark. The small amount of MTS reduced for the zero glyoxylate control was subtracted from that obtained in the presence of glyoxylate.

Chemical Production of Glyoxylate

Glyoxylate is a product of base-catalyzed N-dealkylation of carbinolamides. Incubation of 2.5 mM α-hydroxyhippurate in 1.0M NaOH for 12 hours at 37° C. resulted in the conversion of α-hydroxyhippurate to benzamide as determined by reverse-phase HPLC. The resultant glyoxylate concentration was determined via the malate synthase/malate dehydrogenase couple after appropriate dilution with H₂O to a final glyoxylate concentration of <40 μM.

Enzymatic Production of Glyoxylate

Hippurate (N-benzoylglycine, C₆H₅—CO—NH—CH₂—COO⁻) is a PAM substrate that is oxidatively converted to benzamide and glyoxylate. Hippurate oxidation at 37° C. was initiated by the addition of peptidylglycine α-amidating monooxygenase, (0.6 mg) 100 mM MES pH 6.0, 2.0 μM Cu(NO₃)₂, 10 mM ascorbate, and 3.5 mM hippurate in a final volume of 0.5 ml. At 10 min intervals over a period of 110 minutes, 45 μl aliquots were removed and added to 10 μl of 6% (v/v) trifluoroacetic acid to terminate the reaction. Percent conversion of hippurate to benzamide was determined at each time interval by reverse-phase HPLC.

Approximately 20 nanomoles of glyoxylate was removed from the HPLC vials and added to a 0.9 ml solution that contained necessary components for the glyoxylate assay excluding the enzyme couple and MTS/PMS. Ascorbate was eliminated from all samples, prior to glyoxylate determination, with 10 min incubation in the presence of 12 units of ascorbate oxidase at 37° C. Following ascorbate removal the addition of 100 ul enzyme couple and PMS/MTS brought the assay to a final volume of 1 ml which contained 100 mM TEA-HCl pH 7.8, 10 mM MgCl₂, 400 μM acetyl-CoA, 500 μM NAD⁺, 6 U/ml malate synthase, 6 U/ml malate dehydrogenase, and 12 units ascorbate oxidase. The glyoxylate concentration was determined by measuring the absorbance increase at 490 nm after incubation at 37° C. for 1 hr. The amount of glyoxylate and benzamide produced are shown in Table 1. A control for this experiment was performed at each time point, and contained no hippurate.

All glycolate oxidase reactions were performed in Phosphate Buffer pH 7.8, 0.1 mM FAD (fluorescent assay), or 0.1 mm FMN (luminescent assay) with 0.48 U/assay of enzyme. The glyoxal oxidase reaction was carried out at pH 8.0 in 100 mM TEA Buffer with 1 U/ml HRP. Other details are outlined in the figures.

Screening and Identification of α-amidated Peptide

Mouse At-t20 cells, known to secrete mouse Joining Peptide-Gly (mJP-Gly), were grown in the appropriate cell culture medium to 70% confluency, cells were collected and resuspended in fresh medium containing 2 μM disulfiram, a known PAM inhibitor, and incubated for 15 hours for accumulation of mJP-Gly. Spent medium was collected, acid extracted with 0.1% TFA, and desalted prior to RP-HPLC analysis. The desalted extract was lyophilized and resuspended in 200 μl of 0.1% TFA/0.001% Triton-X, prior to HPLC analysis. 50 μl aliquots were injected onto a C18 RP-HPLC column equipped with a quaternary solvent delivery system. Peptides were separated with a gradient elution of 0.1% TFA/ACN over the timespan of 65 minutes at a flow rate of 1.0 ml/min. Samples containing the internal standard were spiked with 2.5 nanomoles mJP-Gly prior to RP-HPLC separation. One minute fractions were collected over the 65 minute separation, lyophilized and treated with PAM. PAM condition utilized were 40 mM MES pH 6.3, 1 mM Ascorbate/Catechol, 0.5 μM CuSO4, 50 U/ml HRP, 15 U/ml PAM the reaction was carried out in a volume of 300 μl for 3 hours at 398 K. Fractions were then brought to 600 μl in 100 mM Sodium Phosphate pH 7.8, and 0.24 u/ml Glycolate Oxidase was added, the reaction persisted for one hour at 398 K. Luminescent detection was carried out utilizing 1 mM Luminol, 1 mg/ml HRP, in Sodium Carbonate Buffer pH 10.5. Fractions whose fate was to be tested by MALDI-TOF were simply lyophilized after RP-HPLC separation, resuspended in 200 μl 0.1% TFA and analyzed.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1

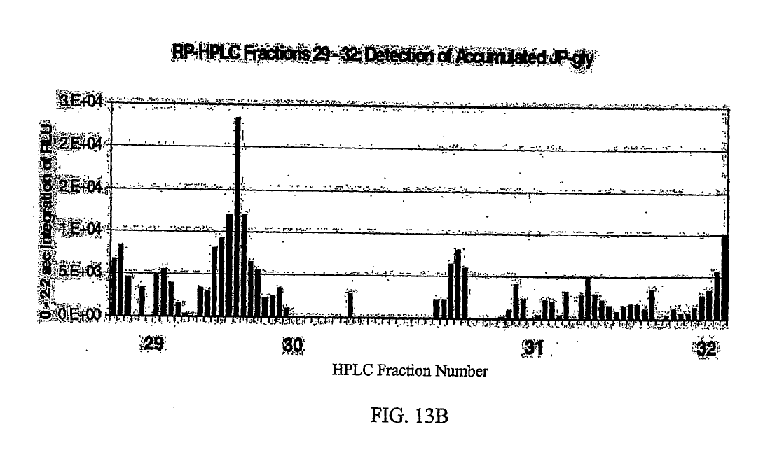

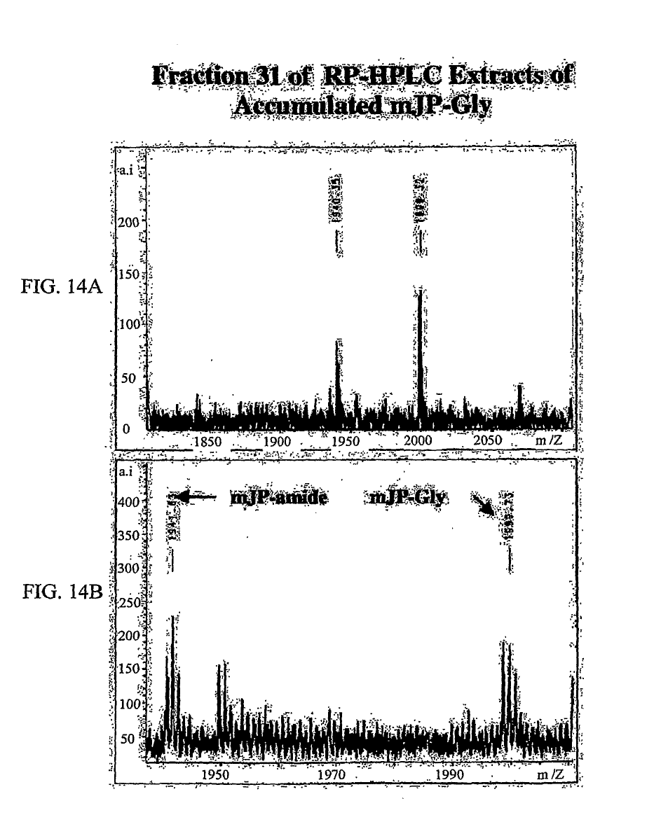

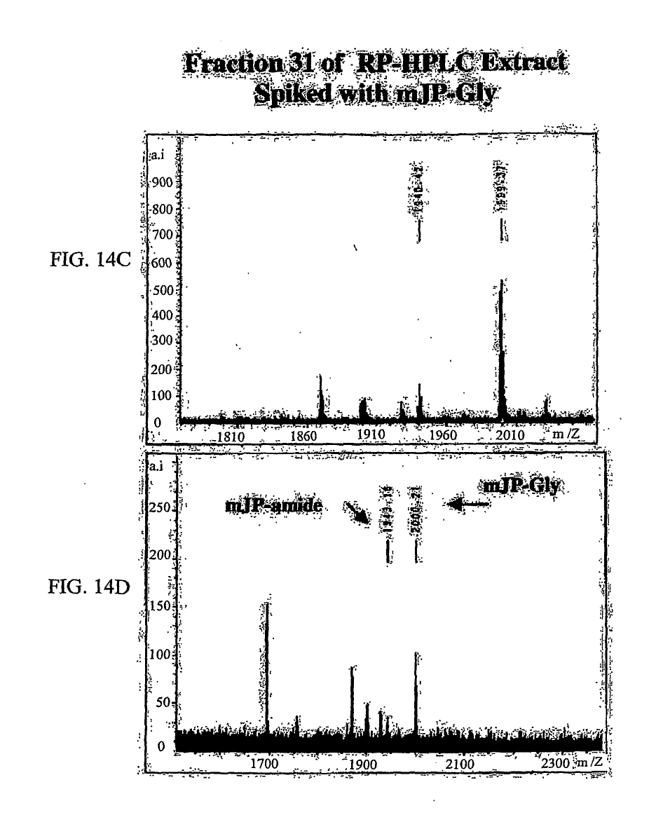

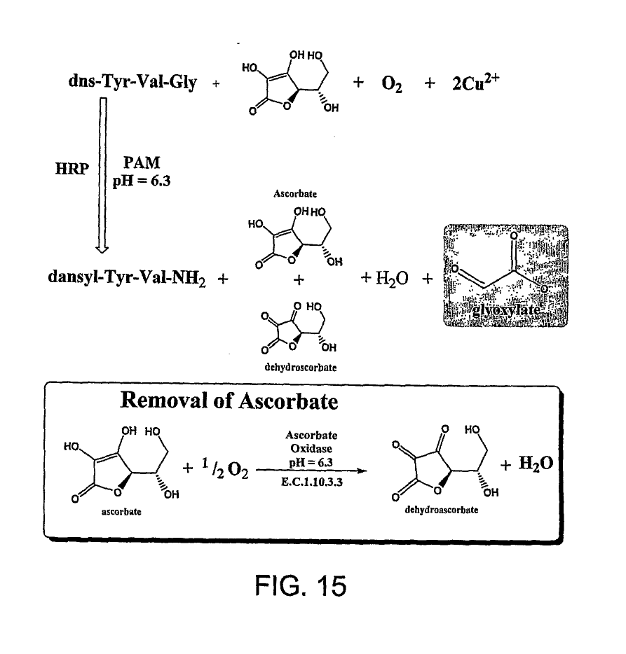

Based on sensitivity alone the luminescent enzyme assay utilizing glycolate oxidase was chosen for application of the glyoxylate assay as a route to the identification of an α-amidated peptide. Any of the assays of the present invention could be used; however, the most sensitive of these techniques is more desirable. A cell line of mouse pituitary cells known to express mouse joining peptide (mJP) (Ala-Glu-Glu-Glu-Ala-Val-Trp-Gly-Asp-Gly-Ser-Pro-Glu-Pro-Ser-Pro-Arg-Glu-Gly) were grown in cell culture to approximately 80% confluency. Cells were then grown in the presence of a PAM inhibitor in order to accumulate the glycine-extended peptides. Spent media was fractionated by Reverse-Phase High Performance Liquid Chromatography (RP-HPLC), and each fraction was then treated with PAM. Fractions positive for glyoxylate were analyzed against a sample containing an internal standard of mJP, to conclude the glyoxylate assay was indeed correct at identifying the presence of a glycine-extended/α-amidated peptide. To further the analysis, a separate set of fractions which did not undergo the PAM reaction were analyzed by MALDI TOF Mass Spectrometry for the presence of the mJP. Demonstration of the glycine-extended form of mJP being present in the same fraction as glyoxylate proves that both the cell culture PAM reaction was indeed inhibited thus allowing the formation of glyoxylate upon treatment with PAM. All data demonstrated that glyoxylate, mJP-Gly, and mJP-amide were present in the same fraction (see FIGS. 13A-13B and 14A-14D).

TABLE 1
Ratio of [Glyoxylate] Produced to [Benzamide]
Produced by the PAM Treatment of Hippurate

| | Glyoxylate | Benzamide | |
|---|---|---|---|
| Time | Produced (mM) | Produced (mM) | [Glyoxylate]/[Benzamide] |
| 40 | 0.69 | 0.58 | 1.2 |
| 50 | 0.67 | 0.70 | 0.96 |
| 60 | 0.71 | 0.80 | 0.89 |
| 70 | 0.75 | 0.90 | 0.83 |
| 80 | 0.77 | 0.98 | 0.79 |
| 90 | 1.3 | 1.1 | 1.2 |
| 100 | 1.3 | 1.2 | 1.1 |
| 110 | 1.3 | 1.3 | 1.0 |

Average ± standard deviation = 1.0 ± 0.16
Note:
Reactions were initiated by the addition of PAM to 2.5 mM hippurate. At the indicated time, an aliquot was removed and assayed for benzamide by HPLC and glyoxylate using the malate synthase/malate dehydrogenase/MTS/PMS system.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### References

Gragera, R. R. et al. (2000) “Localization of Glyoxylate Dehydrogenase and Glyoxylate Complex Molecules in the Rat Prefrontal Cortex: enzymohistochemical and Immnuocytochemical Study” J. Neurosci. Res. 59:561-567.
Asplin, J. R. (2002) “Hyperoxaluric Calcium Nephrolithiasis” Endocrinol. Metab. Clin. North Am. 31:927-949.
Williams, H. E. (1989) “Oxalate Synthesis, Transport and the Hyperoxaluric Syndromes: J. Urol. 141:742-749.
Albrecht, A. M. et al. (1962) “Determination of Aliphatic Aldehydes by Spectrophotometry” Anal. Chem. 34:398-400.
Soda, K. et al. (1973) “Spectrophotometric Determination of Glyoxylic Acid with o-aminobenzaldehyde and Glycine, and its Application to Enzyme Assay” Agr. Biol. Chem. 37:1393-1400.
Bongers, J. et al. (1992) “Semisynthesis of Human Growth Hormone-Releasing Factors by α-Amidating Enzyme Catalyzed Oxidation of Glycine-Extended Precursors” Peptide Res. 5:183-189.
Kramer, D. N. et al. (1959) “Quantitative Determination of Glyoxylic Acid” Anal. Chem. 31:250-252.
Vogels, G. D. et al. (1970) “Differential Analyses of Glyoxylate Derivatives” Anal. Biochem 33:143-157.
Spikner, J. E. et al. (1962) “Fluorometric Microdetermination of Alpha-Keto Acids” Anal. Chem. 34:1468-1471.
Zarembski, P. M. et al. (1965) “The Fluorometric Microdetermination of Glyoxylic Acid in Blood, Urine and Bacterial Extracts” Biochem. J. 96:218-223.
McFadden, B. A. et al. (1960) “The Determination of Glyoxylic Acid in Biological Systems” Anal. Biochem. 1:240-248.
Nishijima, S. et al. (2001) “Glyoxylate Determination in Rat Urine by Capillary Electrophoresis” Int. J. Urol. 8:S63-S67.
Garcia, A. et al. (2001) “Measurement of Nephrolithiasis Urinary Markers by Capillary Electrophoresis” J. Chromatogr. B 755:287-295.
Funai, T. et al. (1986) “High-Performance Liquid Chromatographic Determination of Glyoxylate in Rat Liver” J. Biochem 99:579-589.
Mentasi, E. et al. (1987) “High-Performance Liquid Chromatographic Determination of Glyoxylic Acid and other Carboxyl Compounds in Urine” J. Chromatogr. B. 417:253-260.
Petrarulo, M. et al. (1988) “High-Performance Liquid Chromatographic Determination of Glyoxylic Acid in Urine” J. Chromatogr. 432:37-46.
Lange, M. et al. (1994) “Fast Method for the Simultaneous Determination of 2-Oxo Acids in Biological Fluids by High-Performance Liquid Chromatography” J. Chromatogr. B 662:97-10

1. A method for detecting glyoxylate in a sample, said method comprising contacting said sample with one or more reagents, wherein the reaction of glyoxylate in said sample with said reagents results in the production of a detectable reaction product; and detecting said detectable reaction product.

2. The method according to claim 1, wherein said detectable reaction product is hydrogen peroxide.

3. The method according to claim 1, wherein said detectable reaction product is detected visually or using a fluorescent assay, a luminescent assay, or a spectrophotometric assay.

4. A method for detecting glyoxylate in a sample, said method comprising:
a) contacting said sample with acetyl-CoA, malate synthase, and malate dehydrogenase, wherein NADH is produced if glyoxylate is present in said sample;
b) detecting said NADH produced, wherein said NADH corresponds to the presence of glyoxylate in said sample.

5. The method according to claim 4, wherein said NADH is detected using phenazine methosulfate (PMS) and a tetrazolium compound.

6. The method according to claim 5, wherein the reaction product produced using phenazine methosulfate and a tetrazolium compound is a reduced formazan product that is detected spectrophotometrically, optionally at about 490 nm.

7. The method according to claim 5, wherein said tetrazolium compound is 3-(4,5-dimethylthiazol-2-yl)-5-(3-carboxymethoxylphenyl)-2-(4-sulfophenyl)-2H-tetrazolium, inner salt (MTS).

8. The method according to claim 4, wherein step a) further comprises contacting with NAD⁺.

9. A method for detecting glyoxylate in a sample, said method comprising:
a) contacting said sample with lactate dehydrogenase and glycolate oxidase, whereby glycolate and hydrogen peroxide are produced if glyoxylate is present in said sample; and
b) detecting said hydrogen peroxide produced in the presence of glyoxylate.

10. The method according to claim 9, wherein said hydrogen peroxide is detected using MBTH and DMAB and a peroxidase.

11. The method according to claim 10, wherein the reaction product produced using MBTH and DMAB and a peroxidase is an indamine dye that is detected spectrophotometrically, optionally at about 590 nm.

12. The method according to claim 9, wherein said hydrogen peroxide is detected using a peroxidase and a substrate that reacts in the presence of said peroxidase to produce a fluorescent molecule, wherein said substrate is optionally Amplex Red, whereby Amplex Red is oxidized to Resorufin in the presence of hydrogen peroxide.

13. The method according to claim 10, wherein said peroxidase is horseradish peroxidase.

14. A method for detecting glyoxylate in a sample, said method comprising:
a) contacting said sample with glycolate oxidase or glyoxal oxidase, whereby oxalate and hydrogen peroxide are produced if glyoxylate is present in said sample;
b) detecting said hydrogen peroxide produced from step a).

15. The method according to claim 14, wherein said hydrogen peroxide is detected using MBTH and DMAB and a peroxidase.

16. The method according to claim 15, wherein said reaction product produced using MBTH and DMAB and a peroxidase is detected spectrophotometrically, optionally at about 590 nm.

17. The method according to claim 14, wherein said hydrogen peroxide is detected using a peroxidase and a substrate that reacts in the presence of said peroxidase to produce a fluorescent molecule, wherein said substrate is optionally Amplex Red, whereby Amplex Red is oxidized to Resorufin in the presence of hydrogen peroxide.

18. The method according to claim 14, wherein said hydrogen peroxide is detected visually or using a fluorescent assay, a luminescent assay, or a spectrophotometric assay.

19. The method according to claim 14, wherein said hydrogen peroxide is detected by contacting said sample with luminol and detecting luminescence.

20. The method according to claim 19, wherein said contacting step is performed at a basic pH and in the presence of a catalyst.

21. The method according to claim 20, wherein said catalyst is horseradish peroxidase.

22. The method according to claim 15, wherein said peroxidase is horseradish peroxidase.

23. The method according to claim 17, wherein said method further comprises contacting said sample with flavin adenine dinucleotide (FAD).

24. The method according to claim 14, wherein said method is performed at a basic pH.

25. The method according to claim 24, wherein said pH is about 8.0.

26. A method for detecting glyoxylate in a sample, said method comprising:
a) contacting said sample with glyoxylate reductase and NADPH, whereby glycolate and NADP⁺ is produced if glyoxylate is present in said sample; and
b) detecting said NADP⁺.

27. The method according to claim 26, wherein said NADP⁺ is detected spectrophotometrically by measuring absorbance of said sample, optionally at about 340 nm.

28. A method for assaying for a glycine extended molecule in a sample, wherein said method comprises:
a) contacting said sample with PAM, wherein glyoxylate is produced if a glycine extended molecule is present in said sample; and
b) assaying for the presence of glyoxylate, whereby the presence of glyoxylate is indicative of the presence of a glycine extended molecule.

29. The method according to claim 28, wherein the presence of glyoxylate is assayed using a method selected from:
i) contacting said sample with one or more reagents, wherein the reaction of glyoxylate in said sample with said reagents results in the production of a detectable reaction product; and detecting said detectable reaction product; or,
ii) a) contacting said sample with acetyl-CoA, malate synthase, and malate dehydrogenase, wherein NADH is produced if glyoxylate is present in said sample: and
b) detecting said NADH produced, wherein said NADH corresponds to the presence of glyoxylate in said sample; or,
iii) a) contacting said sample with lactate dehydrogenase and glycolate oxidase, whereby glycolate and hydrogen peroxide are produced if glyoxylate is present in said sample; and
b) detecting said hydrogen peroxide produced in the presence of glyoxylate; or,
iv) a) contacting said sample with glycolate oxidase or glyoxal oxidase, whereby oxalate and hydrogen peroxide are produced if glyoxylate is present in said sample; and
b) detecting said hydrogen peroxide produced; or,
v) a) contacting said sample with glyoxylate reductase and NADPH, whereby glycolate and NADP⁺ is produced if glyoxylate is present in said sample; and
b) detecting said NADP⁺ produced.

30. The method according to claim 28, wherein said glycine extended molecule is a glycine extended peptide.

31. The method according to claim 28, wherein said glycine extended molecule is a glycine extended fatty acid.

32. The method according to claim 28, wherein said glycine extended molecule is a glycine extended prohormone.

33. The method according to claim 28, wherein said glycine extended molecule is an N-acyl-glycine or an N-aryl-glycine conjugated molecule.

34. The method according to claim 28, wherein step (a) further comprises contacting said sample with ascorbate and/or hydrogen peroxide.

35. The method according to claim 28, wherein step (a) further comprises contacting said sample with catechol and/or hydrogen peroxide.

36. The method according to claim 28, wherein after step (a) said sample is contacted with ascorbate oxidase.

37. A method for detecting peptidylglycine α-amidating monooxygenase (PAM) in a sample, said method comprising detecting the presence of glyoxylate using a method selected from:
i) contacting said sample with one or more reagents, wherein the reaction of glyoxylate in said sample with said reagents results in the production of a detectable reaction product and detecting said detectable reaction product; or,
ii) a) contacting said sample with acetyl-CoA, malate synthase, and malate dehydrogenase, wherein NADH is produced if glyoxylate is present in said sample; and
b) detecting said NADH produced, wherein said NADH corresponds to the presence of glyoxylate in said sample; or,
iii) a) contacting said sample with lactate dehydrogenase and glycolate oxidase, whereby glycolate and hydrogen peroxide are produced if glyoxylate is present in said sample; and
b) detecting said hydrogen peroxide produced in the presence of glyoxylate; or,
iv) a) contacting said sample with glycolate oxidase or glyoxal oxidase, whereby oxalate and hydrogen peroxiderroduced if glyoxylate is present in said sample; and
b) detecting said hydrogen peroxide produced; or,
v) a) contacting said sample with glyoxylate reductase and NADPH, whereby glycolate and NADP⁺ is produced if glyoxylate is present in said sample; and
b) detecting said NADP⁺ produced,
wherein the presence of glyoxylate is indicative of the presence of PAM.

38. A method for assaying for the presence of a glycine extended substrate, said method comprising detecting the presence of peptidylglycine α-amidating monooxygenase (PAM) according to the method of claim 37, wherein the presence of PAM activity is indicative of the presence of a glycine extended substrate, and assaying for the presence of a glycine extended substrate in the sample that is determined to contain PAM.

39. A method for screening for the presence of an α-amidated peptide or fatty acid in a sample, said method comprising:
a) growing cells in a medium in which said cells will grow;
b) preparing cell extract and/or obtaining spent media from the cells grown in step a);
c) chromatographically fractionating said cell extract and/or spent media from step b);
d) contacting the fractionated samples obtained from step c) with peptidylglycine α-amidating monooxygenase;
e) assaying said fractionated sample for the presence of glyoxylate using a method selected from:
i) contacting said sample with one or more reagents, wherein the reaction of glyoxylate in said sample with said reagents results in the production of a detectable reaction product; and detecting said detectable reaction product; or,
ii) a) contacting said sample with acetyl-CoA, malate synthase, and malate dehydrogenase, wherein NADH is produced if glyoxylate is present in said sample; and
b) detecting said NADH produced, Wherein said NADH corresponds to the presence of glyoxylate in said sample; or,
iii) a) contacting said sample with lactate dehydrogenase and glycolate oxidase, whereby glycolate and hydrogen peroxide are produced if glyoxylate is present in said sample: and
b) detecting said hydrogen peroxide produced in the presence of glyoxylate; or
iv) a) contacting said sample with glycolate oxidase or glyoxal oxidase, whereby oxalate and hydrogen peroxide are produced if glyoxylate is present in said sample; and
b) detecting said hydrogen peroxide produced; or,
v) a) contacting said sample with glyoxylate reductase and NADPH whereby glycolate and NADP is produced if glyoxylate is present in said sample; and
b) detecting said NADP produced,
wherein fractions that test positive for glyoxylate are indicative of the presence of an α-amidated peptide or fatty acid.

40. The method according to claim 39, wherein said cell medium comprises an inhibitor of PAM.

41. The method according to claim 39, wherein said method further comprises characterizing said detected peptide by mass spectrometry.

42. The method according to claim 39, wherein said method further comprises determining the amino acid sequence of said detected peptide.

43. The method according to claim 1, wherein said method comprises contacting said sample with ascorbate.

44. The method according to claim 43, wherein said method further comprises contacting said sample with ascorbate oxidase after contact with ascorbate.

45-56. (canceled)

57. The method according to claim 4, wherein said method comprises contacting said sample with ascorbate.

58. The method according to claim 57, wherein said method further comprises contacting said sample with ascorbate oxidase after contact with ascorbate.

59. The method according to claim 9, wherein said method comprises contacting said sample with ascorbate.

60. The method according to claim 59, wherein said method further comprises contacting said sample with ascorbate oxidase after contact with ascorbate.

61. The method according to claim 14, wherein said method comprises contacting said sample with ascorbate.

62. The method according to claim 61, wherein said method further comprises contacting said sample with ascorbate oxidase after contact with ascorbate.

63. The method according to claim 26, wherein said method comprises contacting said sample with ascorbate.

64. The method according to claim 63, wherein said method further comprises contacting said sample with ascorbate oxidase after contact with ascorbate.

65. A kit comprising in one or more containers:
acetyl-CoA; malate synthase; and malate dehydrogenase; or
lactate dehydrogenase; and glycolate oxidase; or
glycolate oxidase or glyoxal oxidase; a peroxidase; and a substrate that reacts in the presence of said peroxidase to produce a fluorescent molecule, wherein said substrate is optionally Amplex Red; or
glyoxylate reductase.

66. The kit according to claim 65, further comprising:
phenazine methosulfate; and a tetrazolium compound.

67. The kit according to claim 66, wherein said tetrazolium compound is 3-(4,5-dimethylthiazol-2-yl)-5-(3 -carboxymethoxylphenyl)-2-(4-sulfophenyl)-2H-tetrazolium, inner salt (MTS).

68. The kit according to claim 65, further comprising: NAD⁻.

69. The kit according to claim 65, further comprising:
3-methyl-2-benzothiazolinone hydrazone (MBTH); 3-(dimethylamino)benzoic acid (DMAB); and a peroxidase.

70. The kit according to claim 65, further comprising:
flavin adenine dinucleotide (FAD) or flavin mononucleotide (FMN).

71. The kit according to claim 65, further comprising ascorbate and/or ascorbate oxidase and/or catechol and/or PAM.
